(19) United States
(12) Patent Application Publication
(10) Pub. No.: US 2013/0310903 A1
(43) Pub. Date: Nov. 21, 2013

(54) Anti-Depression Light-Wave Device and Usage Thereof

(71) Applicant: Catherine Y. LI, San Jose, CA (US)

(72) Inventor: Catherine Y. LI, San Jose, CA (US)

(21) Appl. No.: 13/848,716

(22) Filed: Mar. 21, 2013

(30) Foreign Application Priority Data
Mar. 21, 2012 (CN) 201210077047.9
Mar. 21, 2012 (CN) 201220109813.0

(51) Int. Cl. A61N 5/06

(52) U.S. Cl. CPC A61N 5/0618
USPC 607/88

(57) ABSTRACT

An anti-depression light-wave device includes a switch module, a control module and a light source module, wherein the switch module is connected to a regulatory module by the control module and the regulatory module is connected to the light source module. It provides a voltage of 2.3-2.5V for the light source module. The light produced by the light source is monochromatic light of visible light. Preferable wave range is 590-630 nm of the orange light. Moreover, a light source is provided. The anti-depression light-wave device is used as a device to prevent and treat depression. A significant improvement effect for depression, reduction of side effects of antidepressants is shown.

### NOTICE OF COPYRIGHT

A portion of the disclosure of this patent document contains material which is subject to copyright protection. The copyright owner has no objection to any reproduction by anyone of the patent disclosure, as it appears in the United States Patent and Trademark Office patent files or records, but otherwise reserves all copyright rights whatsoever.

### BACKGROUND OF THE PRESENT INVENTION

1. Field of Invention

The present invention relates to an optical device, and more particularly to an anti-depression light-wave device and usage thereof.

2. Description of Related Arts

Seasonal Affective Disorder (SAD)/Major Depressive Disorder (MDD)

Depression has become common in the high-pressure society of today. Most people, at some point in their lives, will suffer from sadness, lack of energy, or even loss of self-esteem for an extended period of time. However, when these aforementioned feelings last for several weeks and start to affect a person's daily life this is classified as a major depressive disorder, or MDD (A.D.A.M). There are miscellaneous reasons for this depression disorder. Some data shows that the depression disorder is inheritable (Virginia Commonwealth University), and also often triggered by traumatic environmental events. It has been found that MDD patients cannot produce or retain enough of the monoamine neurotransmitter in their brains, such as serotonin, dopamine, or norepinephrine. Serotonin is suspected to be in charge of regulating other neurotransmitter systems, so a lack of serotonin may cause a low level of norepinephrine as well. All of these monoamine neurotransmitters are considered to play some important roles regarding anxiety, interest, and energy.

Seasonal Affective Disorder (SAD) is a subtype of MDD that afflicts many people during the winter seasons due to the lack of sunlight. The corresponding symptoms include feeling lethargic and sluggish, excessive sleeping, and experience mood swings. However, the causes of SAD may be different from those of MDD. The lack of sunlight is considered as the primary cause. When melanopsin, which is a photoreceptor within the ganglion cells of the retina that regulate the body's circadian rhythm (Lindsley), detect light, the production of the hormone melatonin is inhibited.

Melatonin is a hormone that causes a drop in body temperate and a feeling of sluggishness. It is also responsible for helping to trigger a sleep response when there is no sunlight. When there is a lack of sunlight in the winter months, melatonin continues to be produced throughout the day which results in a continuous state of weariness.

How to Evaluate Depression?

Recent technological developments can make the evaluation of depression increasingly more reliable and accurate. However, many of the above mentioned modem evaluation methods are still undergoing experimental phases. The brain is a very complicated organ, and methods to collect data from the brain are far less developed than other more simple organs.

Electroencephalography, or EEG, is one method that many researches are using. This method includes attaching electrodes on the patient's scalp to record the brain's electrical activity (Healthwise). In terms of evaluating depression, EEGs seem to reveal biomarkers of depression such as decreased delta wave power during the night, shortened REM latency, and frequent sleep interruption according to one study at the Max Planck Institute of Psychiatry (Wake Sleep File). These biomarkers are classical symptoms of a depression individual. Anti-depressants will suppress random eye movement (REM) sleep and lengthen REM latency, which can be an indicator of a drug's effectiveness.

It can be done as an event-related potential where the brain's reaction to stimuli is averaged.

The use of EEGs can be extremely useful in certain circumstances due to its high temporal resolution. Changes over time periods as short as a few milliseconds can be more easily recorded on an EEG than through function magnetic resonance imaging (FMRI) or a computed tomography scan (CT). It has been observed that the amplitude and delay of the P300 wave produced during a decision making process of a depressed individual is different from that of a non-depressed individual. The P300 wave is an electrical reaction of the brain related to decision making, categorization, and evaluation processes. Event-related potential EEGs have revealed the difference in the P300 wave between a depression individual and a non-depressed individual can vary as much as 250-600 milliseconds after stimulation. In one study, the amplitude of this wave in depressed individuals was shown to be smaller than that of a non-depressed individual at around a range of 4-5 microvolts. Once drugs were administered to these depression individuals their P300 wave returned to a normal amplitude.

EEGs can often times be unreliable due to the sheer amount of data picked up, blinking and head movement all serve to create noise, as well as the millions of operations performed by the brain every second. Even until now scientists are still searching for clear EEG biomarkers for diseases such as MDD.

Functional magnetic resonance imaging, or FMRI, serves a similar purpose, and recent studies have centered on its use as a predictor of drug treatment response. A FMRI operates by detecting changes in blood flow throughout the brain, and using blood-oxygen level as an indicator to measure neuronal activity. Its high spatial resolution has allowed it to become a front-line tool for the diagnosis of tumors and more visible neurological conditions.

Most clinics still rely on the use of standardized e questionnaires in order to diagnose and evaluate patients with depressive symptoms because the nature of the disorder is still so unclear. Generally, both self-administered and doctor-administered tests are used including the Zung Self-Rating Depression Scale, the Zung Self-Rating Anxiety Scale, the Hamilton Anxiety Rating Scale, and the Hamilton Depression Rating Scale. An average of these surveys helps to form a reliable overview of the severity of a patient's depression.

How to Treat Depression?

A combination of anti-depressant drugs and psychotherapy is the most common clinical treatment for individuals who suffer from depression. 70% to 80% of patients have had significant changes after drug treatment. Anti-depressants can be grouped into several categories such as selective serotonin reuptake inhibitors (SSRI), serotonin norepinephrine reuptake inhibitors (SNRI), tricyclics, and monoamine oxidase inhibitors (MAOI). SSRIs, SNRIs, and MAOIs all work by blocking the cleanup of their respective monoamines, while tricyclics increase the amount of these neurotransmitters rather than attempting to prevent their reuptake. SSRIs tend to cause the least side effects, and are therefore the front-line treatment for most cases. However, all anti-depressants do carry side effects which include nausea, insomnia, and potentially suicidal thoughts. Anti-depressants typically take two to four weeks to take effect.

Paroxetine, also known as PAXIL, is a widely used orally administered SSRI with a standard dose of 20-80 mgs. It is used to treat depression, anxiety, and obsessive compulsive disorders. There are a variety of side effects associated with PAXIL, but the most common are quite mild which include headaches.

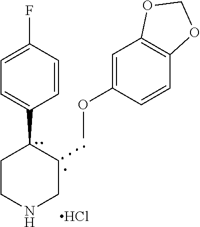

The above is the chemical structure of Paroxetine.

Aside from anti-depressant drugs, another form of effective treatment is light therapy. The intensity of light and wave length are both important. For example, the illumination of an average living room is about 100 lux, while direct sunlight can range from 5000 to 20000 lux, so therefore sunlight can be over 50 times more intense than a normal light bulb. Moreover, it is found that the wave length of light has a strong influence on the efficacy of light therapy. Scientists also research on how to use light to treat not only SAD, but also MDD. One study has explored the benefits of using green light therapy to speed up the effects of citalopram to modest success.

Light therapy works by stimulating the melanopsin photoreceptors located in the ganglion cells of the retina. Exposure to light will cause the melanopsin to reduce production of melatonin, a hormone that causes drowsiness in the absence of light. This causes a patients energy level to be elevated and to modify their biological clock to fit a normal day-night cycle.

Light therapy works through stimulation of melanopsin, the photopigment that regulates the body's circadian rhythm on the retinal ganglion cells, to inhibit the depression cause by a lack of light. Since melanopsin is the most sensitive to blue light, this is the wavelength of light that is typically used in clinics.

High energy white light (10000 lux) was formally used in light therapy, but the low-energy short waves, while shown to be effective, and result in the following:

“Retinohypothalamic tract can be activated by light without melanopsin. It is shown that the assisted effect of rod cells or cone cells, moreover, rod cells or cone cells are more sensitive to green light.

Corresponding interpolating data of fitting curves shows that a 470-480 nm blue peak. If melanopsin, rod cells, and cone cells are all concerned in light simulation, the single opsin-curve will not be the correction explanation.”

Green light also seems to be a promising form of treatment, as it can stimulate both cones and melanopsin. The effectiveness of green light under lower energy can reduce the side effects of eyestrain, headaches, and insomnia associated with light therapy.

Currently there are no studies that investigate orange light (590nm) light for treatment for SAD.

### SUMMARY OF THE PRESENT INVENTION

The invention is advantageous in that it provides an anti-depression light-wave device and usage thereof to overcome the disadvantages of the prior art.

The technical problem solved by the present invention is through the technical methods as follows:

One aspect of the present invention provides an anti-depression light-wave device, which comprises a switch module, a control module, and a light source module, wherein the switch module is connected to a power source and the control module simultaneously, and the control module is connected to the light source module.

In addition, the anti-depression light-wave device further comprises a regulatory module, wherein the switch module is connected to the regulatory module by the control module, and the regulatory module is connected to the light source module to provide a voltage of 2.3 to 2.5V for the light source module.

The light source further comprises a light source which produces a light and a light source shade, wherein the light produced by the light source is a monochromatic wavelength of visible light. Preferably the light source can produce a green light with a wave range of 492-577 nm. Preferably the light source can produce an orange light with a wavelength of 590-630 nm.

The light source comprises:

a. a first light source: LED;

b. a second light source: LED plus fluorescent powder

c. a third light source: LED plus quantum dots(s);

d. a fourth light source: incandescent blub; or

e. a fifth light source: either alone of high pressure sodium lamp(s) or in combination with other light sources;

wherein the switch module comprises at least one switch button of the light source, at least one light intensity button and at least one time of light button, wherein the switch module further comprises: a remote controller, wherein the remote controller is connected to the control module through infrared communication, wherein the control module is a converter, a printed circuit board (PCB), a programmable logic controller (PLC) or a central processing unit (CPU), wherein the the regulatory module is a load resistance.

The second aspect of the present invention is to provide a light source for an anti-depression device, wherein the light produced by the light source is monochromatic wavelength of visible light. Preferably, the light source produces orange light with a wavelength of 590-630 nm, wherein the light source is comprised of:
a. a first light source: LED;
b. a second light source: LED plus fluorescent powder;
c. third light source: LED plus quantum dot(s);
d. a fourth light source: incandescent bulb; or
e. a fifth light source: either alone with high pressure sodium lamps or in combination with other light sources.

The third of the present invention provides a usage of an anti-depression light-wave device, wherein the anti-depression light-wave device is used to prevent and treat depression.

The fourth aspect of the present invention provides a usage of an anti-depression light-wave device, wherein the anti-depression light-wave device is used to prevent and treat anxiety.

The fifth aspect of the present invention provides a method of using an anti-depression device, wherein the anti-depression device is able to be used for 30 to 180 minutes per day to output 250 to 1000 lux of green and/or orange light.

Advantages of the present invention are as follows:

a. Light therapy improves a patient's depression symptoms.

b. The anti-depression device can be sued for a long term to reduce the long term side effects of anti-depressant drugs.

c. The anti-depression device provided by the present invention has a simple structure, has low manufacturing cost, is inexpensive, is able to improve depression and the patient's quality of life.

Additional advantages and features of the invention will become apparent from the description which follows, and may be realized by means of the instrumentalities and combinations particular point out in the appended claims.

Still further objects and advantages will become apparent from a consideration of the ensuing description and drawings.

These and other objectives, features, and advantages of the present invention will become apparent from the following detailed description, the accompanying drawings, and the appended claims.

In accordance with another aspect of the invention, the present invention comprises

### BRIEF DESCRIPTION OF THE DRAWINGS

FIG. 1 is the schematic diagram of the present invention.

FIG. 2 is the circuitry schematic diagram of one embodiment of the present invention.

FIG. 3 is the structure schematic diagram of the present invention.

FIG. 4A is the light analysis spectrum of the green light.

FIG. 4B is the light analysis spectrum of the white light.

FIG. 4C is the light analysis spectrum of the orange light.

FIG. 5 is the result of SDS.

FIG. 6 is the result of HAMD.

FIG. 7 is the result of SAS.

FIG. 8 is the result of HAMS.

FIG. 9 is the color temperature distribution diagram data cited from www.mediacollege.com

FIG. 10 is event-related electroencephalogram of depression patients, with a P300 wave of 1.2 μV.

FIG. 11 is event-related electroencephalogram of healthy adults with a P300 wave of 4.6 μV.

### DETAILED DESCRIPTION OF THE PREFERRED EMBODIMENT

The following description is disclosed to enable any person skilled in the art to make and use the present invention. Preferred embodiments are provided in the following description only as examples and modifications will be apparent to those skilled in the art. The general principles defined in the following description would be applied to other embodiments, alternatives, modifications, equivalents, and applications without departing from the spirit and scope of the present invention.

Experiment methods without detailed terms of the following embodiments are the normal conditions, or conditions provided by the producer.

### Embodiment 1 Anti-Depression Light-Wave Device

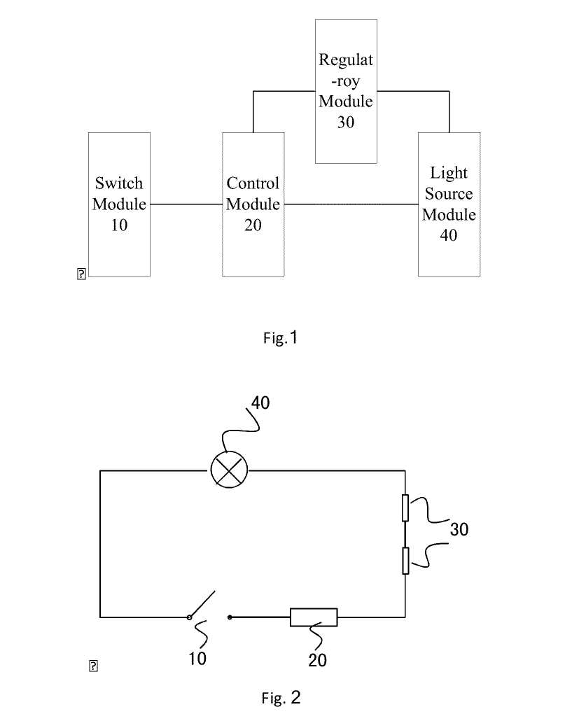

An anti-depression light-wave device, referring to FIG. 1, comprises a switch module 10, a control module 20, a light source module 40 and a body 50, wherein the switch module 10 is connected to the power supply and the control module simultaneously, and the control module is connected to the light source module 40.

The body 50 is a rectangle made of plastic, wherein the switch module 10 is arranged on the top of the body 50.

The switch module comprises a switch button of the light source 110, light intensity buttons 120, and time of light button 130, wherein the switch button of the light source 110 is in able to control activation and deactivation of the light source module 40.

The control module is a printed circuit board, or PCB. The control module contains a microprocessor with control software to control the intensity and time of the light through a PWM adjusting luminance mode. The intensity of the light can be selected from compatible intensities using the intensity light button 120 from 250 to 1000 lux.

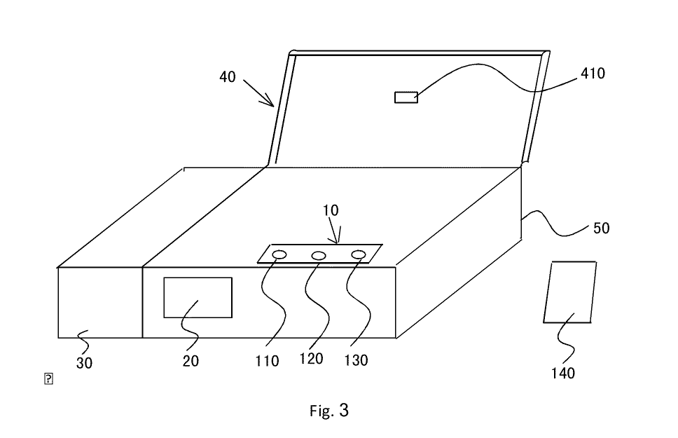

In FIG. 3, the light source module is arranged on the top body 50, and the light source module 40 is hinged at the body 50. The light source module 40 comprises a light source 410 and a light source shade. The light source shade is constructed of a transparent material.

The light source is an LED that produces an orange light with a wavelength of 597 to 630 nm, and can also add fluorescence power or quantum power on the LED.

### Embodiment 2 Anti-Depression Light-Wave Device

An anti-depression light-wave device, referred to in FIG. 1, FIG. 2, and FIG. 3 comprises a switch module 10, a control module 20, a regulatory module 30, a light source module 40, and a body 50, wherein the switch module 10 is connect to the regulatory module 30 through the control module 20, wherein the regulatory module 30 is connected to the light source module 40, and the regulatory module 30 provides a voltage of 2.3 to 2.5V for the light source module 40.

The body 50 is a rectangle made of plastic, and the switch module 10 Is arranged on the top of body 50.

The switch module 10 comprises a switch button of the light source 110, light intensity button 120, and time of light button 130. Furthermore, the switch module can be applied as a remote controller 140, wherein the remote controller 140 is connected of the control module 20 through an infrared communication sensor. Moreover, the switch module 10 can be adapted as a display screen or a touch screen.

The control module is a programmable logic controller, or PLC, or a central processing unit, or CPU.

The regulatory module has an adapted load resistance, an input resistance of 500 KΩ minimum, and an output resistance of 10 KΩ.

Referring to FIG. 3, the light source module 40 is arranged on the top of the body 50, wherein the light source module 40 is hinged at the body 50. The light source module 40 comprises the light source 410 and a light source shade, wherein the light source shade is made of a transparent material.

The light source 410 produces an orange light with a wavelength of 590-630 nm (wave range), and can also can add fluorescence power or quantum power to the LED.

Embodiment 3 Light Source of Anti-Depression Light-Wave Device

A light source of an anti-depression light-wave device producing monochromatic wavelengths of visible light. Preferably, the light source is able to produce a green light with a wavelength of 492-577 nm. Preferably, the light source is able to produce orange light with a wavelength of 597-622 nm. Furthermore, the wavelength of the orange light is 607.5 nm.

The light source can be:

a. a first light source: LED;

b. a second light source: LED plus fluorescent powder;

c. a third light source: LED plus quantum dot(s);

d. a fourth light source: incandescent bulb; or

e. a fifth light source: high pressure sodium lamp

A method of using the anti-depression light-wave device able to be used 30 to 180 minutes per day with a intensity of 250 to 1000 lux, and able to produce green and/or orange light

### Embodiment 4 Effectiveness of the Anti-Depression Light-Wave Device

Evaluating the effective of light therapy with different wavelengths of light.

Green light with a wavelength of 526 nm, orange light with a wavelength of 607.5 nm, and white light all of which having a light-energy of 250-1000 lux was administered to patients suffering from depression with a 20 mg dose of paroxetine. The was conducted as a double-blind randomized controlled trial to ensure reliable results

Two parents were administered 30 minutes of green light therapy per day for two weeks. Then this therapy was combined with paroxetine for four additional weeks.

Four evaluation tables including the Zung Self-Rating Anxiety Scale (SAS), the Zung Self-Rating Depression Scale (SDS), Hamilton Anxiety Rating Scale (HAMS) and Hamilton Depression Rating Scale (HAMD). Using the above data to compare the reduction of the Anxiety and Depression Index before and after the light therapy. The orange and green light are compared against light therapy using white light at the same light-energy level to compare the effectiveness between the orange and green light therapy.

Method Procedure

1. Materials

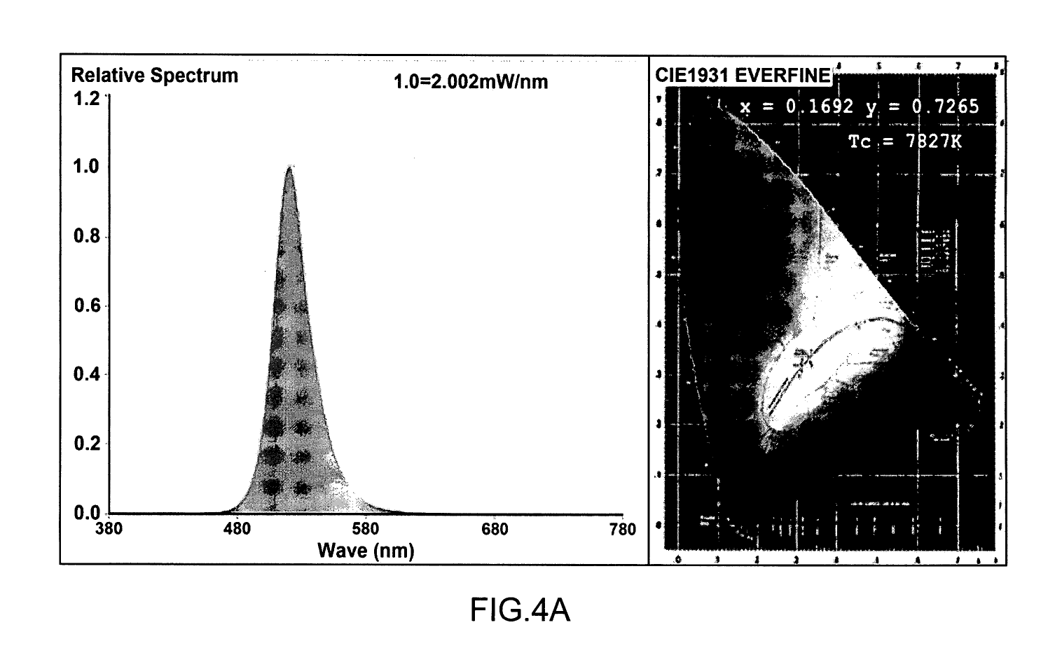

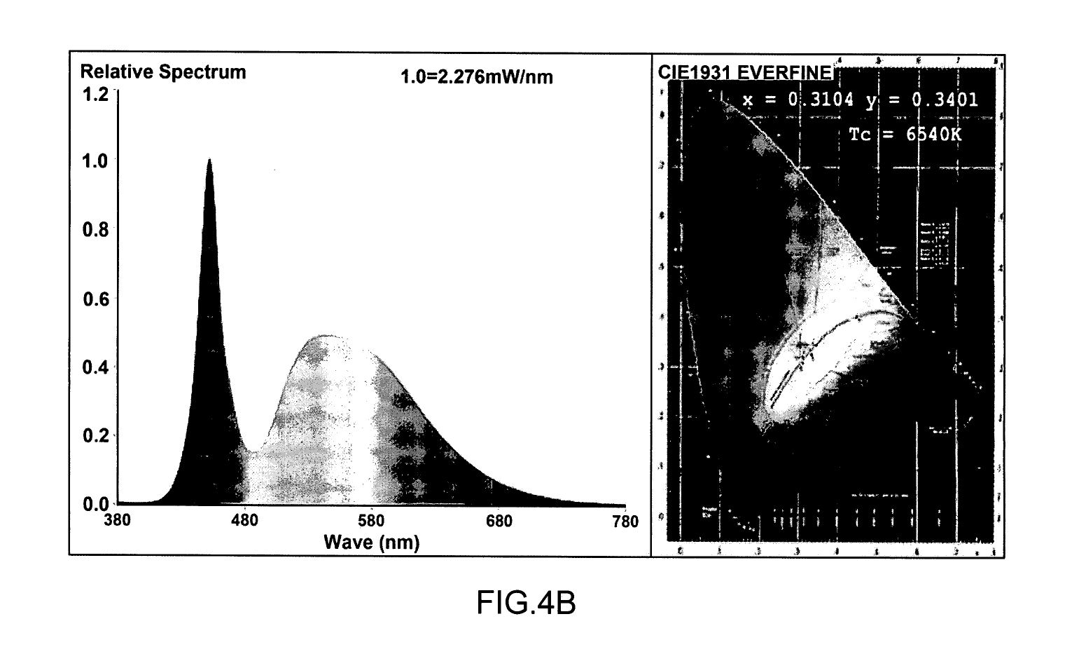

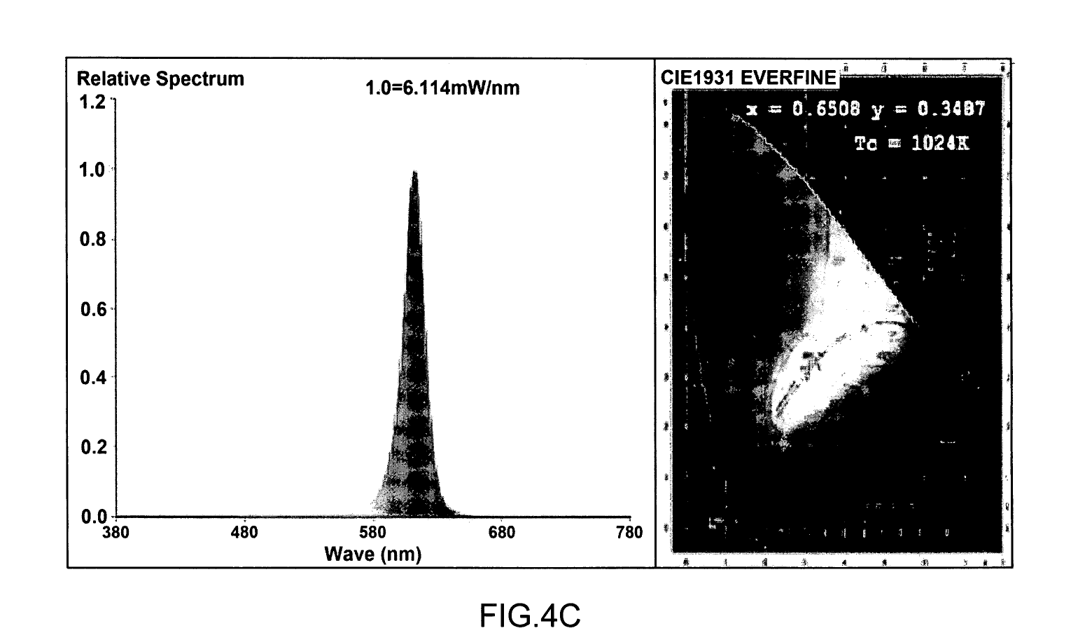

Referring to the light source analysis data in FIG. 4A, FIG. 4B, FIG. 4C, FIG. 4A is the spectrum analysis of the green light. FIG. 4B is the spectrum analysis of the white light. FIG. 4C is the spectrum analysis diagram of the orange light. The results are presented in Table 1.

TABLE 1
Test Monochromatic Lights

| Color of 15 × 7.5 cm | Peak wavelength | Intensity | Color temperature |
|---|---|---|---|
| light therapy device | (nm) | (lux) | (kelvins) |
| Green | 527.0 | 260.38 | 7827 |
| White | 495.5 | 472.2 | 6540 |
| Orange | 607.0 | 325.39 | 1024 |

2 g paroxetine (each patient was administered 20 mg per day)

Zung Self-Rating Anxiety Scale (SAS)

Zung Self-Rating Depression Scale (SDS)

Hamilton Anxiety Rating Scale (HAMS)

Hamilton Depression Rating Scale (HAMD)

2 Participants

(1) Participants

The participants included in this study were primarily of East Asian descent. Participants were outpatients seeking treatment for depressive symptoms at the Shanghai Mental Health Center. Participants had to show mild to moderate symptoms of depression on the SAD, SDS, HAMD, and HAMS questionnaire. They had to have no prior history of taking any anti-depressants. Gender and age varied depending on availability, but participants ages in this experiment ranged from 18 to 55.

(2) Risk Assessment

Possible side effect risks of light therapy included eyestrain, headaches, and insomnia, but patients were informed of this prior so as to avoid looking directly at the light sources. Risks were also minimized by the light brightness on the devices. The risk of being deprived of normal drug treatment was accounted for, as patients continued to receive 20 mg of paroxentine per day. It is worth mentioning that the side effects of paroxetine include nausea and weight gain.

(3) Protection of the Participants

In regards to the protection of privacy, the data collection was not anonymous. This was due to the fact that patients were required to return to the mental health center to collect data twice. In regards to a notify risk, participants were asked to sign an informed letter of consent stating they were informed of the purpose and risk.

3. Method: Double-Blind Experiment

Participants were randomly sorted into three groups. Each group received green IS or orange light therapy. Each participant was evaluated based on the SAS, SDS, HAMD, and HAMS before the start of their trials.

Each participant paid a small deposit and took home a light therapy device to be used from 7:00 AM to 9:00 AM every weekday for the first two weeks. The device was to be placed 15 cm from the face.

These devices were numbered and packaged to conform to a double-blind study. The student researches and supervisors had a table that described the order in which each of the lights would be distributed (on a first come first serve basis, in the interest of randomness) for the purposes of data analysis. It is worth mentioning that the doctors did not know which light each patient was receiving. Patients took a daily prescription of 20 mg of paroxentine as a low-dose SSRI treatment for the entirety of the four week trial period.

At the end of the two week experiment, participants returned to the mental health center to return their light therapy devices and provide a SAS, SDS, HAMS, and HAMD questionnaire for a second time.

At the end of four weeks into the experiment, participants returned to the mental health center to provide a SAS, SDS, HAMS, and HAMD questionnaire for a third time.

4. Results

Statistical analysis results are shown in Table 2.

TABLE 2
Changes in Depression/Anxiety Index before and after the Experiment and T-Test

| | SAS | | | | | | | | | |
|---|---|---|---|---|---|---|---|---|---|---|
| | | | T critical | | | | | T critical | | |
| | | | value 95% | | | | | value 95% | | |
| | Avg. change | Number of | confidence | T | [symbol] ificance | Avg. change | Number of | confidence | T | [symbol] ificance |
| | after 2 weeks | patients | interval | value | test | after 4 weeks | patients | interval | value | test |
| Green | 3.5 | 2 | 6.3 | 1.48 | No | 3.5 | 2 | 6.3 | 1.48 | No |
| light | | | | | | | | | | |
| White | 0.5 | 2 | 6.3 | 0.21 | No | −7 | 1 | Sample value | 2.09 | N/A |
| light | | | | | | | | too small | | |
| Orange | 7.67 | 3 | 2.9 | 3.97 | Yes | −8 | 2 | 6.3 | 3.38 | No |
| light | | | | | | | | | | |
| | SDS | | | | | | | | | |
| | | | T critical | | | | | T critical | | |
| | | | value 95% | | | | | value 95% | | |
| | Avg. change | Number of | confidence | T | [symbol] ificance | Avg. change | Number of | confidence | T | [symbol] ificance |
| | after 2 weeks | patients | interval | value | test | after 4 weeks | patients | interval | value | test |
| Green | −8.5 | 2 | 6.3 | 4.23 | No | −3 | 2 | 6.3 | 1.86 | No |
| light | | | | | | | | | | |
| White | −4.5 | 2 | 6.3 | 2.24 | No | −3 | 1 | Sample value | 1.05 | N/A |
| light | | | | | | | | too small | | |
| Orange | −11.33 | 3 | 2.9 | 6.91 | Yes | −9 | 2 | 6.3 | 5.59 | No |
| light | | | | | | | | | | |
| | HAMA | | | | | | | | | |
| | | | T critical | | | | | T critical | | |
| | | | value 95% | | | | | value 95% | | |
| | Avg. change | Number of | confidence | T | [symbol] ificance | Avg. change | Number of | confidence | T | [symbol] ificance |
| | after 2 weeks | patients | interval | value | test | after 4 weeks | patients | interval | value | test |
| Green | −5.5 | 2 | 6.3 | 4.62 | No | −2.5 | 2 | 6.3 | 2.11 | No |
| light | | | | | | | | | | |
| White | −1 | 2 | 6.3 | 0.84 | No | −3 | 1 | Sample value | 1.86 | N/A |
| light | | | | | | | | too small | | |
| Orange | −5.67 | 3 | 2.9 | 5.85 | Yes | −7.5 | | 6.3 | 6.30 | Yes |
| light | | | | | | | | | | |
| | HAMD | | | | | | | | | |
| | | | T critical | | | | | T critical | | |
| | | | value 95% | | | | | value 95% | | |
| | Avg. change | Number of | confidence | T | [symbol] ificance | Avg. change | Number of | confidence | T | [symbol] ificance |
| | after 2 weeks | patients | interval | value | test | after 4 weeks | patients | interval | value | test |
| Green | −7.5 | 2 | 6.3 | 7.43 | Yes | −2 | 2 | 6.3 | 1.98 | No |
| light | | | | | | | | | | |
| White | −2.5 | 2 | 6.3 | 2.48 | No | −4 | 1 | Sample value | 2.80 | N/A |
| light | | | | | | | | too small | | |
| Orange | −6 | 3 | 2.9 | 7.23 | Yes | −9.5 | 2 | 6.3 | 9.41 | Yes |
| light | | | | | | | | | | |

[symbol]

indicates data missing or illegible when filed

T critical values are at a 95% confidence interval (6.3 when n=2; 2.9 when n=3)

There was no difference between the initial depression/anxiety scores and subsequent scores.

Note: An additional two patients “2 week scores” were recorded (white and orange respectively), but were omitted due to incompleteness. They were consistent with these trends, however the averages varied by less than one.

The data was analyzed by finding the change in depression/anxiety index for each individual questionnaire, and each subsequent score was compared with the initial one, and then the mean change for all the patients of the same color was found.

With the mean change for each color known, the student's T-test was performed for each data point. The normal range of SAS scores was 29.78±10.06. This means that 10.06 was 3 standard deviations, and thus the standard deviation of the SAS was by dividing 10.06 by 3 to obtain 3.35 A similar method was used for SDS (normal range=33.46±8.55), and a standard deviation was calculated to be 2.85 The standard deviation of HAMS and HAMD were calculated to be 1.68 and 1.43 respectively based on those of the SAS and the SDS> The following equation was used to find the Z value:

$$Z = \frac{\overline{X}}{\frac{σ}{\sqrt{n}}}$$

Z values of less than one were highlighted in yellow in the above table as they signified a statistically insignificant change. Z values greater than the 95% confidence interval critical value were highlighted in green, as they signified that a change in values before and after the experiments. For critical values of 6.3, the probability of Type II error was 5% for n=2. For critical values of 2.9, the probability of Type II error was 5% for n=3.

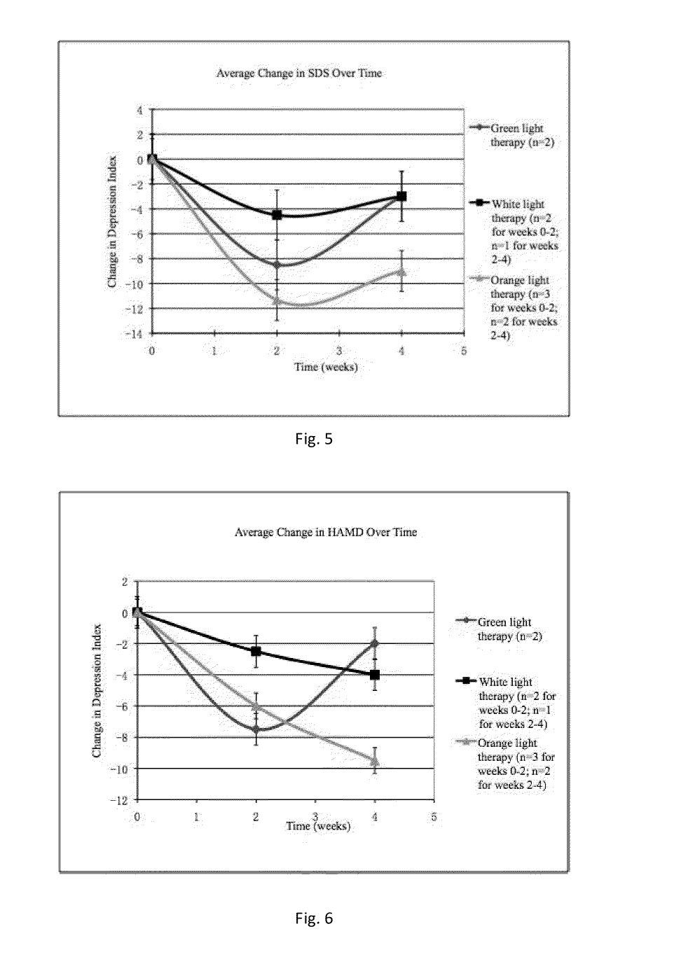

Changes of depression and anxiety are referred to in FIG. 5 to FIG. 8.

FIG. 5 shows the SDS results, green light (n−2), white light (n−1), white light 2 weeks, 2 patients, 4 weeks, 1 patient, orange light (n−2), orange light 2 weeks, 3 patients, 4 weeks, and 2 patients.

FIG. 6 shows the HAMD results, green light (n−2), white light (n−1), white light 2 weeks, 2 patients, 4 weeks, 1 patient, orange light (n−2), orange light 2 weeks, 3 patients, 4 weeks, and 2 patients.

FIG. 7. shows SAS results, green light (n−2), white light (n−1), white light 2 weeks, 2 patients, 4 weeks, 1 patient, orange light (n−2), orange 2 weeks, 3, patients 4 weeks, and 2 patients.

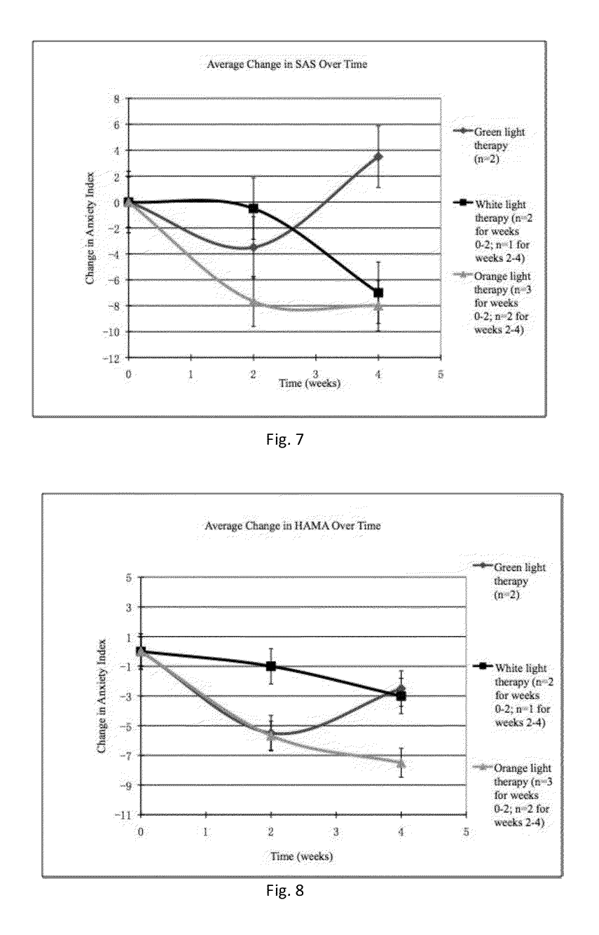

FIG. 8 shows HAMS results, green light (n−2), white light (n−1), white light 2 weeks, 2 patients 4 weeks, 1 patient, orange light (n−2), orange 2 weeks, 3 patients, 4 weeks, 2 patients.

5. Conclusion

As hypothesized, orange and green light therapy caused significant changes across all four questionnaires in the first two weeks, compared to the negligible improvement for those using placebo white light.

It was also hypothesized that the green light would perform better than orange light therapy. Instead, orange light seemed to provoke larger changes than green light on each questionnaire in week 1, with the exception of the HAMD. The difference was not significantly large, so more data and an in depth study are required to verify this.

Orange light has not been studied in-depth, so these are promising preliminary findings. The reason for its effectiveness may be that orange has a much warmer color temperature than green light, and this is one of the main distinguishing factors between overcast skylight and that of sunny days. Orange light may also share some of the physical benefits of red light therapy.

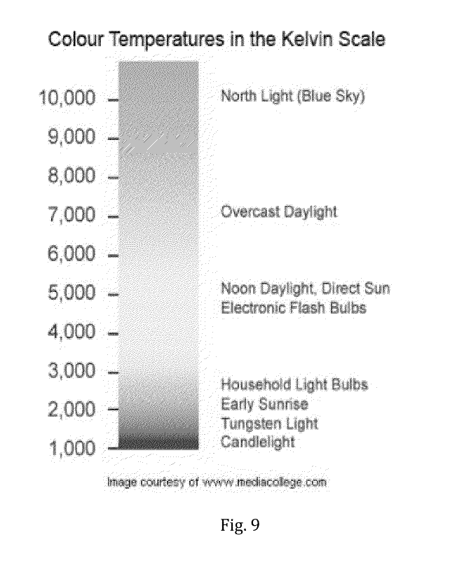

FIG. 9 is a color temperature distribution diagram with data cited from www.mediacollege.com.

It is interesting to note that after four weeks, the green and orange light therapy exhibited significant differences. Patients who received green light therapy regressed greatly after two weeks of light therapy had been removed and the paroxetine took effect. Patients who received orange light therapy stayed relatively steady in comparison.

Another possible reason for this may be that the high-energy waves of green light may provide too much stimulation, and that the body's circadian rhythm has difficulties readjusting once the light is removed. This is more applicable in the cases of anxiety, where heightened energy levels can exacerbate the problem, and this is seen enormously in the amount of regression of the SAS patients.

Few definitive conclusions may be drawn, however, this investigation has some limitations. The sample size was extremely limited, as factors such as medical history were tightly controlled in order to enhance the quality of the study. Individual patients tend to react very differently to different treatment methods; only 65% of patients settle with their first drug prescription. The study also involved participants taking light devices home for personal use each morning, so it is impossible to verify whether or not they actually used it and if so properly. And the low availability of participants made it difficult to use age to gender-matched controls.

Future Studies

With such intriguing preliminary data, there is a large amount of potential for future study. The next logical step would likely be to continue collecting data, especially that of orange light therapy-treated patients, so as to increase the statistical validity of the study. Age or gender-matched controls would, if possible, also be an excellent expansion. If these same trends remain visible, orange light could prove an innovative new solution to a common mental disorder. Post-therapy regression could also be an avenue as it would help researchers understand the mechanisms of SAD. And a trial done without antidepressant drug assistance, if possible, would serve to strengthen the results.

Furthermore, the inclusion of electroencephalography as a quantitative evaluator of depression would be an exciting expansion. Some EEG data was collected in the past months that was intended for use in this study, but due to the lack of resources it was left incomplete despite showing encouraging results.

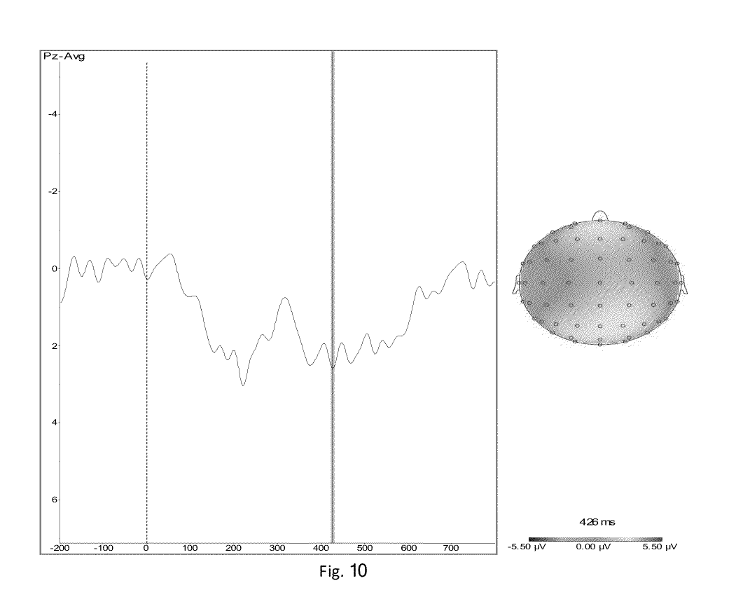

FIG. 10 is event-related electroencephalogram of depression patients with a P300 wave of 1.2 μV.

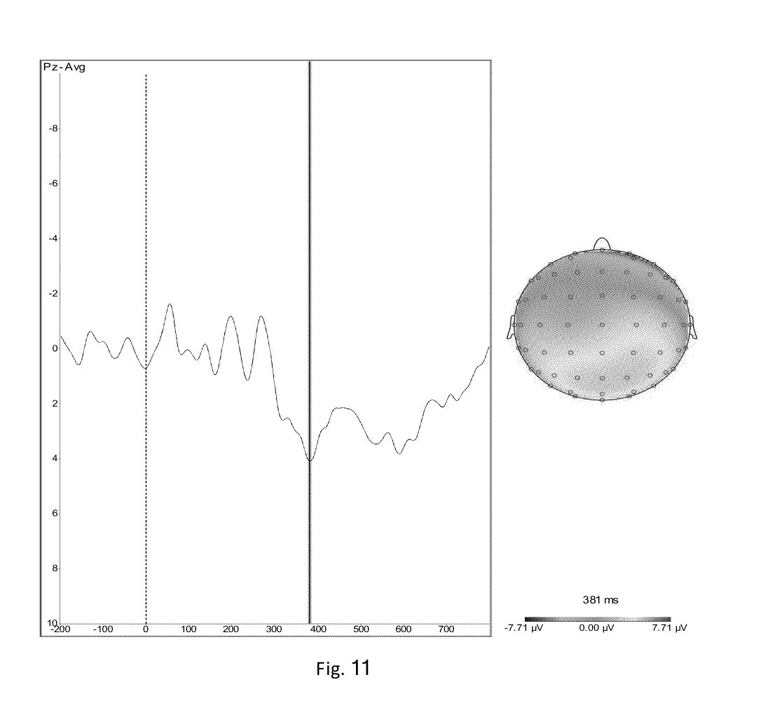

FIG. 11 is event-related electroencephalogram of healthy adults with a P300 wave of 4.62 μV.

This EEG data, as shown above, indicates a large difference in P300 waveforms between depressed and non-depressed individuals. Reportedly, medication can eliminate some of this difference. If light therapy affects EEG waveforms in the same manner as drugs, this could confirm whether or not these P300 waves are true biomarkers, or conversely whether or not light and medication changes something fundamental about the brain, rather than a physical side effect of anti-depressants.

Additional Remarks:

Participants of the present invention were less because the number of patients of the first treatment without drug administration background varies less. The patients had misgivings after being diagnosed with depression and anxiety if they were dispensed drugs. Therefore, the present invention adopted a treatment of light therapy plus drugs of a minimum dosage.

Table 3 provides Clinical Global Impression (CGI) results of Paroxetine treatment group with outcome classifications of 12 weeks administration.

TABLE 3
Paroxetine treatment group CGI with 12-week administration

| Outcome | Placebo | Paxil 20 mg | Paxil 40 mg | Paxil 60 mg |
|---|---|---|---|---|
| Worse | 14% | 7% | 7% | 3% |
| No Change | 44% | 35% | 22% | 19% |
| Minimally | 24% | 33% | 29% | 34% |
| Much Improved | 11% | 18% | 22% | 24% |
| Very Much | 7% | 7% | 20% | 20% |

The present invention is shown to have a significant improvement for individuals suffering from depression with a reduction in side effects caused by anti-depressants, and therefore the light therapy has wide applications in the future to improved a depressed patient's quality of life.

One skilled in the art will understand that the embodiment of the present invention as shown in the drawings and described above is exemplary only and not intended to be limiting.

It will thus be seen that the objects of the present invention have been fully and effectively accomplished. The embodiments have been shown and described for the purposes of illustrating the functional and structural principles of the present invention and is subject to change without departure from such principles. Therefore, this invention includes all modifications encompassed within the spirit and scope of the following claims.

One skilled in the art will understand that the embodiment of the present invention as shown in the drawings and described above is exemplary only and not intended to be limiting.

It will thus be seen that the objects of the present invention have been fully and effectively accomplished. The embodiments have been shown and described for the purposes of illustrating the functional and structural principles of the present invention and is subject to change without departure from such principles. Therefore, this invention includes all modifications encompassed within the spirit and scope of the following claims.

### CITED REFERENCE

A.D.A.M. “Major depression.” A.D.A.M. Medical Encyclopedia. U.S. National Library of Medicine, 11 Mar. 2011. Web. 6 Mar. 2012. <http://www.ncbi.nlm.nih.gov///>.

Francesco, Benedetti, et al. “Morning light treatment hastens the antidepressant effect of citalopram: a placebo-controlled trial.” PubMed. US National Library of Medicine, 2003. Web. 12 Mar. 2012 <http://www.ncbi.nlm.nih.gov///?dopt=Abstract&holding=f1000,f1000m srctn>.

Ganghadar, B. N., et al. “P300 amplitude in non-bipolar, melancholic depression.” Journal of Affective Disorders 28.1 (1993): 57-60. ScienceDirect. Web. 12 Mar. 2012. <http://www.sciencedirect.com////W>.

Gooley, Joshua J., et al. “Spectral Responses of the Human Circadian SystemDepend on the Irradiance and Duration of Exposure to Light.” Science TranslationalMedicine. Science Translational Medicine, 12 May 2010. Web. 3 Jan. 2012. <http://strn.sciencemag.org/Nra33.abstract>.

Gordijn, M. C. M., D., t Mannetje, and Y. Meesters. “The effects of blue-enriched light treatment compared to standard light treatment in seasonal affective disorder.” Journal of Affective Disorders (2011): n. pag. PDF file.

Healthwise. “Electroencephalography(EEG).” WebMD.Healthwise Incorporated, 26 Jul. 2010. Web. 6 Mar. 2012. <http://www.webmd.com//eeg-21508>.

Lam, R W, et al. “The Can-SAD study: a randomized controlled trial of the effectiveness of light therapy and fluoxetine in patients with winter seasonal affective disorder.” PubMed.gov. US National Library of Medicine, May 2006. Web. 8 Mar. 2011. <http://www.ncbi.nlm.nih.gov/pubmed/16648320?dopt=Abstract>.

Lindsley, Gila. “Seasonal Affective Disorder (SAD): About light, depression & melatonin.” Healthy Resources. New Technology Publishing, Inc., 2010. Web. March 8 Loving, Richard T., et al. “Bright green light treatment of depression for older adults.” BMC Psychiatry 5 (2005): n. pag. BioMed Central. Web. 12 Mar. 2012. <http://www.biomedcentral.com/-244X//#B8>.

NASA. “Light emitting diodes bring relief to young cancer patients; NASA technology used for plant growth now in clinical trials.” Marshall Space Flight Center News 5 Nov. 2003: n. pag. NASA. Web. 12 Mar. 2012. <http://www.nasa.gov///////-199.html>.

Nutt, D J. “Relationship of neurotransmitters to the symptoms of major depressive disorder.” J Clin Psychiatry (2008): n. pag. PubMed. Web. 6 Mar. 2012. <http://www.ncbi.nlm.nih.gov//#>.

Phelps, Jim. “Light Therapies For Depression.” PsychEducation.org. N.p., November 2009 Web. 8 Mar. 2011. <http://www.psycheducation.org/depression/LightTherapy.htm>.

Tracy, Natasha. “Antidepressant Medications for Depression.” Healthy Place. N.p., 14 Jan. 2012. Web. 6 Mar. 2012. <http://www.healthyplace.com///medications-for-depression/>.

Virginia Commonwealth University. “Study Suggests That Heritability Of Major Depression Is Higher In Women Than In Men.” Science Daily. N.p., 6 Jan. 2006. Web. 6 Mar. 2012. <http://www.sciencedaily.com////.htm>.

Wang, Jijun. Personal interview. 3 Jan. 2012.

What is claimed is:

1. An anti-depression light-wave device comprising: a switch module, a control module and a light source module, wherein said switch module is connected to a power and said control module simultaneously, and said control module is connected to said light source module, wherein said light source module comprises a light source which produces light and a light source shade, wherein said light produced by said light source is monochromatic light of visible light.

2. The anti-depression light-wave device, as recited in claim 1, further comprising a regulatory module, wherein said switch module is connected to said regulatory module by said control module; and said regulatory module is connected to said light source module, and provides a voltage 2.2˜2.5V for said light source module.

3. The anti-depression light-wave device, as recited in claim 2, wherein said light source produces a green light with a wave range of 492-577 nm.

4. The anti-depression light-wave device, as recited in claim 2, wherein said light source produces an orange light with a wave range of 590-630 nm.

5. The anti-depression light-wave device, as recited in claim 3, wherein said light source is elected from a group consisting of:
a first light source: LED;
a second light source: LED plus fluorescent powder;
a third light source: LED plus quantum dot;
a fourth light source: incandescent bulb; and
a fifth light source: either alone of high pressure sodium lamp(s) or in combination with other light sources.

7. The anti-depression light-wave device, as recited in claim 6, wherein said switch module further comprises: a remote controller, wherein said remote controller is connected to said control module through an infrared sensing communication.

8. The anti-depression light-wave device, as recited in claim 6, wherein said control module is a converter, a printed circuit hoard (PCB), a programmable logic controller (PLC) or a central processing unit (CPU).

9. The anti-depression light-wave device, as recited in claim 8, wherein said regulatory module is a load resistance.

10. A light source of an anti-depression device producing a light selected from a group consisting of a monochromatic light of visible light and an orange light with a wave range of 590-630 nm.

11. The light source of an anti-depression device, as recited in claim 10, wherein said light source is selected from a group consisting of:
a first light source: LED;
a second light source: LED plus fluorescent powder;
a third light source: LED plus quantum dot;
a fourth light source: incandescent bulb; and
a fifth light source: either alone of high pressure sodium lamp(s) or in combination with other light sources.

12. A usage of an anti-depression light-wave device as recited in claim 1, wherein said anti-depression light-wave device is used as a device to prevent and treat depression.

13. A usage of an anti-depression light-wave device as recited in claim 2, wherein said anti-depression light-wave device is used as a device to prevent and treat depression.

14. A usage of an anti-depression light-wave device as recited in claim 3, wherein said anti-depression light-wave device is used as a device to prevent and treat depression.

15. A usage of an anti-depression light-wave device as recited in claim 4, wherein said anti-depression light-wave device is used as a device to prevent and treat depression.

16. A usage of an anti-depression light-wave device as recited in claim 5, wherein said anti-depression light-wave device is used as a device to prevent and treat depression.

17. A usage of an anti-depression light-wave device as recited in claim 6, wherein said anti-depression light-wave device is used as a device to prevent and treat depression.

18. A usage of an anti-depression light-wave device as recited in claim 7, wherein said anti-depression light-wave, device is used as a device to prevent and treat depression.

19. A usage of an anti-depression light-wave device as recited in claim 8, wherein said anti-depression light-wave device is used as a device to prevent and treat depression.

20. A usage of an anti-depression light-wave device as recited in claim 12, wherein said anti-depression light-wave device is used 30˜180 minutes per day, 250˜1000 lux, and green and/or orange light.
